(19) United States
(12) Patent Application Publication
(10) Pub. No.: US 2009/0253163 A1
(43) Pub. Date: Oct. 8, 2009

(54) ITERATIVE STAINING OF BIOLOGICAL SAMPLES

(75) Inventors: Jun Xie, Niskayuna, NY (US); Fiona Ginty, Saratoga Springs, NY (US); Robert John Filkins, Niskayuna, NY (US); Michael Christopher Montalto, Albany, NY (US); Anup Sood, Clifton Park, NY (US); Jeffrey Bernard Fortin, Niskayuna, NY (US); Wei-Cheng Tian, Clifton Park, NY (US); Michael John Gerdes, Albany, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: GENERAL ELECTRIC COMPANY, Schenectady, NY (US)

(21) Appl. No.: 12/061,044

(22) Filed: Apr. 2, 2008

(51) Int. Cl. G01N 1/30; C12M 1/34; C12M 1/36

(52) U.S. Cl. 435/40.5; 435/287.1; 435/288.7; 435/286.5

(57) ABSTRACT

Automated methods and devices that facilitate iterative staining of biological samples from imaging applications are provided. The methods include the steps of providing a small volume flow cell containing a biological sample, applying a stain to the biological sample, combining at least two precursor reagents to form an activated destaining agent and wherein the activated destaining agent decomposition rate is greater than or similar to the destaining reaction rate, and flowing the destaining agent over the biological sample at a flow rate that is greater than the decomposition rate of the activated destaining agent. The process of staining, combining and flowing may be iteratively repeated. Also disclosed herein are devices for iterative staining of biological samples comprising a flow cell, in fluid communication with a premixer, wherein the volume capacity of the premixer is smaller than about five times the volume capacity of the flow cell.

### BACKGROUND

For multiplexed applications, tissue samples or tissue microarrays (TMA) need to be stained with multiple molecular probes to investigate protein expression or spatial distribution quantitatively or qualitatively. The staining process is typically performed using time-consuming manual techniques that are susceptible to error. The reagents used in the staining process are often expensive and have limited shelf life thereby requiring special handling techniques.

Automated systems that use microscopic flow cells as reaction chambers for tissue samples or to monitor cellular activities under flow conditions exist. However, such systems have not been well adapted to use in tissue sample processing, lacking environmental control of the sample within the flow cell and requiring manual intervention.

Fluid flow rates of reagents (e.g., luminescent reagents) through the flow cell is difficult to control as a flow stream may cause turbulence within the small volume chamber, dislodging or damaging the sample. Also, peripheral external heating, (e.g., from a heated microscope stage), may cause non-uniform heating of the enclosed sample. Consequently, the temperature varies across the sample. Further, repeated reagent preparation, sample removal and replacement into the stage for image acquisition, require sample realignment and diminish reproducibility.

### BRIEF DESCRIPTION

The invention generally relates to automated methods and devices that facilitate iterative staining of biological samples from imaging applications.

In some embodiments the methods include the steps of providing a small volume flow cell containing a biological sample, applying a stain to the biological sample, combining at least two precursor reagents to form an activated destaining agent and wherein the activated destaining agent decomposition rate is greater than or similar to the destaining reaction rate, and flowing the destaining agent over the biological sample at a flow rate that is greater than the decomposition rate of the activated destaining agent. The process of staining, combining and flowing may be iteratively repeated.

In some embodiments a device for iterative staining of a biological sample is provided and comprises a flow cell in fluid communication with a premixer, wherein the volume capacity of the premixer is smaller than about five times the volume capacity of the flow cell.

In some embodiment the flow cell comprises a base configured to receive a tissue sample; a thermoelectric element; a gasket position between the base and the thermoelectric element; an inlet port in fluid communication with the premixer; and an outlet port; wherein one or both of the base and thermoelectric elements includes an image acquisition window. The flow cell may further comprise a degasser and a piezo-electric element.

### DESCRIPTION OF THE FIGURES

These and other features, aspects, and advantages of the present invention will become better understood when the following detailed description is read with reference to the accompanying figures wherein.

FIG. 1 illustrates a representative flow cell device.

FIG. 2 illustrates a degasser for use with a flow cell device.

FIG. 3 shows improvement in reaction time of a bleaching reagent using a piezo-electric element as a premixer.

### DETAILED DESCRIPTION

The following detailed description is exemplary and not intended to limit the invention of the application and uses of the invention. Furthermore, there is no intention to be limited by any theory presented in the preceding background of the invention of the following detailed description of the figures.

To more clearly and concisely describe and point out the subject matter of the claimed invention, the following definitions are provide for specific terms, which are used in the following description and the appended claims.

The singular forms “a” “an” and “the” include plural referents unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least each numerical parameter should at least be construed in light of the number of reported significant digits and by applying ordinary rounding techniques

As used herein, the term “biological sample” refers to a sample obtained from a biological subject, including sample of biological tissue or fluid origin obtained in vivo or in vitro. Such samples may be, but are not limited to, tissues, fractions, and cells isolated from mammals including, humans.

As used herein the term “lumiphore” refers to a chemical compound that demonstrates luminescence including chemoluminescence, bioluminescence, phosphorescence, and photoluminescence. Representative examples include, but are not limited to, luminol, lucigenin, acridans, acridinium esters, and dioxetanes, and fluorophores.

As used herein the term “oxidant” or “oxidizing agent” refers to a bleaching reagent that substantially inactivate a lumiphore. Representative oxidizing agents include active oxygen species, hydroxyl radicals, singlet oxygen, hydrogen peroxide, or ozone such as hydrogen peroxide, potassium permanganate, sodium dichromate, aqueous bromine, iodine-potassium iodide, or t-butyl hydroperoxide.

The present invention relates to an automated system and methods that operate with minimal operator intervention by eliminating the need to transfer samples (e.g., tissue samples on a slide within the flow cell). The disclosed systems and methods further eliminate the need to displace samples between the staining component and the imaging component.

Automation of the staining component minimizes both reagent volume and reagent dwell time within the system thereby saving on expensive reagents, such as fluorescence labeled antibodies, and minimizing reagent decomposition or side reactions. It also reduces variations in reagent metering and may reduce occurrences of reagent cross contamination. Automation of the imaging component eliminates or reduces steps associated with image alignment and remounting the sample after staining. The improvement in image registration facilitates formation of an accurate composite image.

Automation may be achieved through computer control of one or more of the process steps involved in sequential staining such as addition of staining reagents and oxidant. Where the flow cell system is incorporated into a combined sample processing and image acquisition system, the image acquisition components (e.g., microscope or camera) may also be controlled by software such as a program written in LabVIEW or C.

### Devices

Provided herein are devices for performing the disclosed methods. Thus, provided herein are flow cells and systems comprising flow cells and premixers.

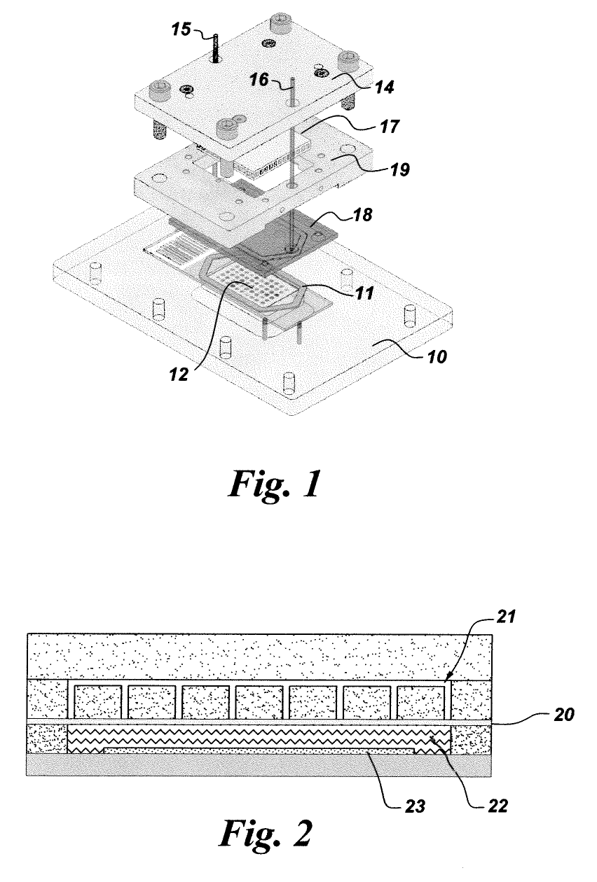

In one embodiment as shown in FIG. 1, a flow cell may comprise an enclosed flow chamber configured to be positioned above a tissue sample. The flow cell may comprise: a solid support-receiving member 10, a gasket 11 with a central opening configured to receive a tissue sample positioned on a slide 12, a lid 14, an inlet port 15, and an outlet port 16, wherein the flow cell defines a closed chamber when a slide is positioned in the slide-receiving member and the gasket is sandwiched between the slide and the lid. As the flow chamber is enclosed inside the flow cell, fluid evaporation and, consequently, reagent loss is minimized. Also, the closed configuration improves temperature control.

The flow cell may be a modular unit that is adapted to fit onto a standard microscope stage. Alternatively, the flow cell may be an integrated unit including a microscope stage. In some embodiments, the flow cell may be fixed on a microscope stage for the imaging process. This allows the sample to be exposed to a complete series of reagents without manual intervention thereby potentially eliminating realignment of the sample on the microscope stage for image acquisition or registration. This is particularly useful for multiplexed staining and imaging as images acquired after each staining step may be superimposed to form a composite image.

The flow cell may be used in a system that includes fluidic and temperature control subsystems to control fluidic delivery and solution temperature in the internal chamber of the flow cell. In one embodiment, the fluidic control system may further comprise reservoirs, flow sensors, mixing chambers, and degassers to prepare one or more reagents prior to injection into the flow cell. The advantage of such a system is to avoid the need of premixing and storing reagents that may have limited stability or shelf life. The fluidic control system is in fluidic communication with the inlet port and outlet port of the flow.

### Slide-Receiving Member

In some embodiments, the flow cell may include a slide-receiving member configured to receive a tissue sample positioned on a solid support such as a glass slide. The slide holder is compatible with a range of chemical and temperature variations. In one embodiment, the slide holder may consist of a base and a pin or tab system for securing the slide in the chamber.

### Gasket

The flow cell includes a gasket with a central opening configured to receive a tissue sample positioned on a slide. The gasket may be made of a deformable, chemically inert, rubber or plastic that retains the liquid applied to the flow chamber. The gasket may optionally include openings for the inlet and outlet ports. The central opening of the gasket maybe sized to maximize the field of view of the image acquisition window. The width, length, and depth of the gasket when placed into the flow cell may each be varied to achieve a predetermined internal volume of the flow cell. In some embodiments, the width and length of the gasket may be sized to conform to standard tissue section slides or microarray substrates. For example, in one embodiment, the central opening of the gasket can accommodate a tissue micro array that is 20 mm wide and 30 mm long.

### Inlet and Outlet Ports

The inlet and outlet ports are preferably placed away from the image acquisition window. Thus, the inlet and outlet ports may be positioned in the gasket or upon the lid. The inlet and outlet ports are typically matched in size such that the in-flow rate and the out-flow rate are coordinated to achieve a desired rate of flow across the sample.

### Temperature Control

Referring further to FIG. 1, the temperature control unit may further comprise a thermoelectric stage 17 for temperature control and an RTD/thermistor for temperature measurement. For configurations wherein the bottom surface of the temperature control unit is in direct contact with the fluid, the contacting surface 18 may be made of chemical resistant material, such as stainless steel or titanium. A frame 19 may also be used to position the components of the temperature control unit.

In some embodiments, the temperature control unit is integrated into the lid so that the internal chamber formed between a temperature control unit (e.g., a Peltier stack) and a slide is heated directly by the temperature control unit, instead of through the tissue slide. This configuration frees up the backside of the tissue slide for imaging.

### Degasser

In some embodiments, the invention further comprises a method for assisting in gas removal from the flow cell. As shown in FIG. 2 a gas permeable film 20 such as an amorphous fluoropolymer or polydimethylsiloxane (PDMS) may be employed to separate gas vent passages 21 and the fluid chamber 22 containing the sample 23. In such configurations application of reverse pressure or a vacuum, vents the system and removes the excessive gas in the chamber.

### Agitation

In some embodiments, the invention may further comprise a piezo-electric element connected to the flow chamber and capable of producing vibration within the flow chamber by conversion of low voltage electrical signals into acoustic energy. In a preferred embodiment the piezo-electric element maybe composed of a ceramic, quartz (SiO2) or barium titanate (BaTiO3). The configuration of the piezo-electric element provides ultrasonic agitation and influences the flow profile of reagents through the fluid chamber. This is particularly advantageous wherein the desired staining reaction is diffusion limited and conventional mechanical mixing is prohibited by the flow cell geometry.

### Computer Processing Units

A computer may control the various components of the flow cell system, including for example the thermal control unit, the premixer, the vibrational unit, and the pumps. Where the flow cell system is incorporated into a combined sample processing and image acquisition system, the image acquisition components (e.g., microscope or camera) may also be controlled by a computer.

### Methods

Also provided herein are methods for processing and acquiring images from a biological sample adhered to a solid support (e.g., a tissue section fixed to a microscope slide). The methods include steps employing various alternative embodiments of the device selected for a particular application. Representative methods for iterative processing of biological samples are described in co-owned U.S. patent application Ser. No. 11/864,093, which is incorporated herein.

One representative method includes: (a) positioning a biological sample, such as a tissue section on a microscope slide, in a flow cell; (b) applying a fluorescent label or a lumiphore to the sample in a manner to allow sufficient contact time between the lumiphore and the sample which are typically in the range of 30 to 60 minutes depending on the concentration and type of label used; (c) applying a wash solution, for example an appropriate buffer solution to wash away any unbound fluorescent label or lumiphore; (d) acquiring an image of the labeled sample; (e) applying a chemical agent to destroy the lumiphore in step (b) by applying an oxidizing agent that substantially inactivates the lumiphore where a solution of the oxidizing agent is applied to the sample using a continuous flow process to minimize non-Laminar flow and dwell time within the flow cell resulting in an average dwell time of 1 to 5 minutes; (f) optionally acquiring an image of the sample, and (g) repeating steps (b)-(f) at least once.

Each of the applying steps may be accomplished by flowing a solution containing a particular reagent over the biological sample positioned within the flow cell. The following parameters may be controlled to enhance reactivity and, thereby, reduce reagent consumption (1) flow cell internal volume; (2) flow cell internal temperature; (3) timing of mixing of constituent parts of the oxidizing solution (e.g., hydrogen peroxide and sodium bicarbonate); (4) extent of agitation of the solutions as they pass the sample; and (5) bubble removal or degassing of the flow cell. Appropriate regulation of these parameters also may reduce sample degradation, permitting a single sample to yield more data.

The automated destaining step permits the operator to reprobe a single sample while maintaining the original registration. The addition of the oxidant results in destaining of the biological sample due to substantial removal of the signal produced by the lumiphore. Whether the destaining is accomplished by chemically altering the lumiphore or by detachment, the signal is reduced by at least 80% and preferably greater than 90%. This reduction in signal may be measured as the post-staining intensity at a particular wavelength relative to the initial absolute intensity of the stained biological to adjust for a concomitant reduction in background signal or autofluorescence resulting from the destaining step.

### Flow Cell Internal Chamber Volume

Small volume flow cells conserve valuable reagents. Where reagent diffusion is the rate-determining step, flow should correlate with the internal chamber volume. For example fluidic delivery to the flow cell may be adjusted based on the volume capacity of the chamber to allow for rapid, complete flushing of the chamber.

The flow cell provides a solid support for the test sample. The flow cell dimensions are constrained based on the solid support used. The height of the flow cell is based on the thickness of the sample. Where the sample is a tissue section, it may have a thickness between about 5 μm to about 100 μm. The tissue section may occupy 20 mm by 30 mm area. This results in a small internal chamber volume in the range of 10 μL to 1000 μL, preferably, 50 μL to 200 μL.

Decomposition may happen before a reagent is substantially removed from the flow cell. Turbulent flow with in the flow chamber improves surface reactivity and facilitates reagent byproducts (e.g., oxygen gas) removal. Accordingly, in some embodiments, the chamber may include an agitation element (e.g., acoustic piezoelectric component) that generates turbulence.

### Internal Chamber Temperature

Although, many microarray staining processes proceed between 20° C. and 100° C., some systems may require significantly higher or lower temperatures with tight tolerance. Adsorption and desorption processes related to staining are temperature dependant and, therefore, in some embodiments, temperature uniformity is provided across the sample surface where the chemical interactions takes place.

For example, a stacked thermoelectric element may be introduced into or upon the chamber wherein current flowing through the elements may regulate chamber temperature, within a specified temperature range, through radiant heating of fluid within the chamber. Some systems may require a temperature tolerance of +/−5° C. while others may have a significantly tighter or less stringent temperature tolerance. In some embodiments, the thermoelectric element may optionally contain a heat sink to absorb and dissipate heat to facilitate temperature regulation.

### Premixer and Mixture Timing

Reagents used in multiplexing staining may have limited shelf life where by effectiveness of the reagents diminishes over time. This occurs when a reagent, produced by mixing two or more solutions to initiate a chemical reaction, may undergo partial decomposition or precipitation. This may lead to the formation of gas and other undesirable by products. In some embodiments, the solutions are completely mix at the molecular level by using a premixer to intersperses the reactants immediately before the reagent is introduced into the flow cell. Mixing times should be sufficient long to generate the reagent and sufficiently limited to prevent decomposition.

The premixer, which is positioned upstream of the flow cell, may be based on a chamber design or a tube design. The chamber design may include a small vessel with inlet and outlet ports and containing a mechanical mixer. The tube design may include a Y-adaptor into which the chemical reagents are driven at a predetermined flow rate. Alternatively, the tube design may include a physical barrier (e.g., a micromesh or a spherical membrane positioned within the tube) or a nozzle that generates turbulence.

The premixer allows for mixing of the chemical reagents before introduction to the flow cell. The volume capacity of the flow cell is determined based on the decomposition rate of the chemical reagents and the desired flow rate of the chemical reagents or their reaction product through the flow chamber.

A peroxide solution decomposition rate at a given temperature equals −dC/dt=kC where C is peroxide concentration, t is time and k is the first order rate constant. Half-life of a first order reaction is independent of the starting concentration and is calculated as t½=In(2)/k. Half-life of nth-order reactions can also be determined and is represented as:

t_{1/2}=2ⁿ⁻¹−1/(n−1)k[A₀]ⁿ⁻¹.

Residence time within the flow cell is limited to less than the half-life of the reagent. Therefore the volume capacity of the flow cell is determined by: Vp<(V/t)(t½) where (Vp) is the volume capacity of the flow cell and (V/t) is the flow rate.

An oxidant such as a hydrogen peroxide solution, which generates hydroperoxide anions, decomposes and forms oxygen gas within 5 minutes of preparation. Typically the volume capacity of the flow cell may range from 1 to 1000 μL, preferably 50 μL to 500 uL. To ensure the average dwelling time of reagents in the flow cell is less than 5 mins, the flow rate preferably ranges from 50 uL/min to 500 uL/min. Applying Vp<(V/t)(t ½), volume capacity of the premixer is limited to 5 to 5000 μL, preferable 250 μL to 2500 μL.

### Reagent Reservoirs

The premixer is in fluid communication with one or more reagent reservoirs. The reagent reservoirs act as storage devices for the reagents prior to deliver to the premixer. A flow controller allows for the transfer of a metered quantity of a reagent to the premixer. The deliver of more than one reagent can be done in a sequential order or in parallel, permitting accurate metering of the reagents and reducing reagent cross contamination.

### Image Acquisition Window

The disclosed methods may be performed in a system that includes a flow cell configured to enable enhanced access to the sample through an image capture window. The image capture window may be defined by the substrate upon which the sample is set (e.g., microscope slide) or may include an optically transmissive material on the underside of the slide-receiving member.

Accordingly, the methods of the invention may be performed using a flow cell in which accessory devices, such as heating elements or agitation elements (e.g. an acoustic piezoelectric component) are positioned away from the image capture window through which a microscope, coupled to a camera, may capture images of the sample during the various phases of processing.

### Controlling Reaction Kinetics

In one embodiment, 3 ml of a hydrogen peroxide buffer solution (3% H₂O₂, pH 10) is prepared in a premixer. The premixer is designed to be in physical communication with the flow cell such that, using continuous flow, the freshly prepared peroxide buffer is introduced into the flow chamber wherein residence time in the chamber is less than 5 mins. A typical flow rate is 250 μL/min and the volume of the flow chamber is less than 250 ul. The chamber further comprises a piezo-electric element.

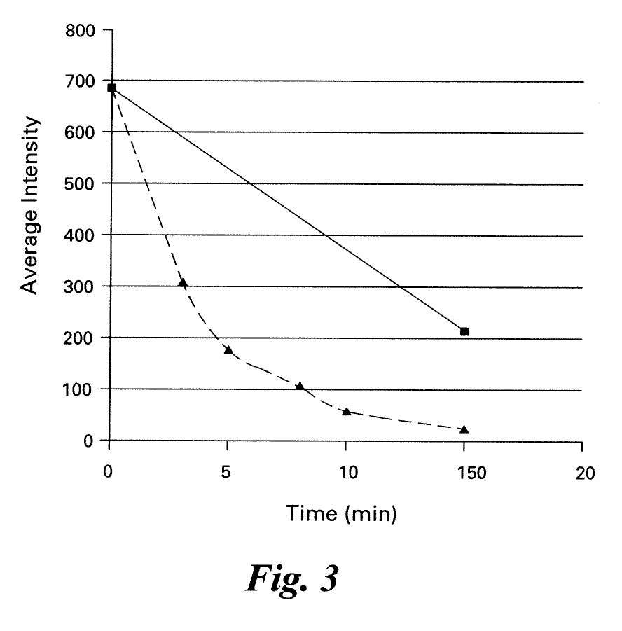

As shown in FIG. 3, these conditions reduce the reaction time about three-fold compared to manual destaining process where the sample is processed in a container and is agitated for about 10 sec for every 5 mins. residence time. Increase reactivity may be attributed to fresh (less decomposed) preparation of the activated destaining agent and the continuous removal of by-products in an equilibrium reaction. In-line premixing and optimal flow rates also reduce the amount of oxygen gas bubbles formed in-situ through the decomposition of hydrogen peroxide in a basic solution.

1. An automated method of iterative staining of a biological sample comprising:
(a) providing a small volume flow cell containing a biological sample;
(b) applying a stain;
(c) combining at least two precursor reagents to form an activated destaining agent, wherein the activated destaining agent decomposition rate is greater than or similar to the destaining reaction rate;
(d) flowing the destaining agent over the biological sample at a flow rate that is greater than the decomposition rate of the activated destaining agent; and
(e) optionally repeating steps (b)-(d) at least once.

2. The method of claim 1, further comprising observing the signal generated by the stained biological sample through an image acquisition window and optionally measuring intensity values of the signal generated and correlating the signal with specific labeling of a biomarker.

3. The method of claim 1, wherein the activated destaining agent includes an oxidant.

4. The method of claim 3, wherein the oxidant is selected from a buffer solution of hydrogen peroxide, aqueous bromine, iodine-potassium iodide, and t-butylhyroperoxide.

5. The method of claim 1, wherein one precursor reagent includes a hydrogen peroxide and another precursor reagent is sodium hydroxide.

6. The method of claim 1, wherein the activated destaining agent is formed in a mixing chamber that is in fluid communication with the flow cell.

7. The method of claim 1, wherein the activated destaining agent is formed by merging flow streams of each precursor reagent through a channel that is in fluid communication with the flow cell and wherein the channel optionally contains a physical barrier to increase mixing of the flow streams of each precursor reagent.

8. The method of claim 1, wherein the stain is a lumiphore.

9. The method of claim 1, further comprising the step of vibrating the flow cell to enhance mixing.

10. The method of claim 9, wherein the vibration is applied using a piezo-electric element attached to the flow cell wherein electrical energy is converted into acoustic energy.

11. The method of claim 1, further comprising increasing the internal temperature of the flow cell above ambient temperature to enhance staining or destaining.

12. The method of claim 1, further comprising reducing gas bubble formation within the flow cell by vacuum venting of the flow cell.

13. The method of claim 1, further comprising transferring the precursor reagents of the activated destaining agent from one or more reagent reservoirs to the premixer, wherein the precursor reagents are delivered to the premixer at predetermined concentrations.

14. The method of claim 1, wherein the one or more of steps (b)-(e) are controlled by a processor.

15. An automated device for iterative staining of a biological sample comprising a flow cell in fluid communication with a premixer, wherein the volume capacity of the premixer is smaller than about five times the volume capacity of the flow cell.

16. The device of claim 15, wherein the volume capacity of the flow cell is between 1 μL to 1000 μL and the volume capacity of the premixer is less than between 5 μL to 5000 μL.

17. The device of claim 15, wherein volume capacity of the flow cell is between 50 μL to 500 μL and the volume capacity of the premixer is less than between 250 μL to 2500 μL.

18. The device of claim 15, wherein the premixer further comprises a pump and a turbulence generator to increase mixing of at least two precursor reagents.

19. The device of claim 18, wherein the turbulence generator is a mixing nozzle, a porous filter, a spherical obstruction, or a micromesh.

20. The device of claim 15, wherein the flow cell comprises a base configured to receive a tissue sample; a thermoelectric element; a gasket position between the base and the thermoelectric element; an inlet port in fluid communication with the premixer; and an outlet port; wherein one or both of the base and thermoelectric element includes an image acquisition window.

21. The device of claim 20, further comprising a degasser to remove gas present within the flow cell.

22. The device of claim 20, further comprising a piezo-electric element attached to the flow cell and wherein the piezo-electric element is capable of producing vibration within the flow cell through conversion of electrical energy into acoustic energy.

23. The device of claim 15, further comprising a reagent reservoir in fluid communication with the premixer.

24. The device of claim 23, further comprising a premixer controller and a reagent flow controller.

25. The device of claim 24, wherein the premixer controller, the reagent flow controller, and optionally a temperature controller attached to the thermoelectric element are integrated with a processor and wherein the processor is configured to control one or more operating parameters of the device.
